(19) United States
(12) Patent Application Publication
(10) Pub. No.: US 2025/0157608 A1
(43) Pub. Date: May 15, 2025

(54) IDENTIFYING POTENTIAL TREATMENT PATHS USING HARMONIC HOMOLOGY FOR DISENTANGLING MULTIWAY DATA INTERACTIONS

(71) Applicant: International Business Machines Corporation, Armonk, NY (US)

(72) Inventors: Filippo Utro, Pleasantville, NY (US); Laxmi Parida, Mohegan Lake, NY (US); Aldo Guzman Saenz, White Plains, NY (US); Davide Gurnari, Warsaw (PL)

(21) Appl. No.: 18/506,194

(22) Filed: Nov. 10, 2023

(51) Int. Cl. G16H 20/00; G06K 7/14

(52) U.S. Cl. CPC G16H 20/00; G06K 7/1413

(57) ABSTRACT

A computer-implemented method includes receiving an input set of multiway data. The multiway data includes a numerical representation of each factor in a set of interconnected factors affecting an outcome and each factor has a codependency on at least one other factor in the set of interconnected factors. A set of persistent homology barcodes is determined based on the multiway data using a processer. At least a first significant persistent homology barcode in the determined set of persistent homology barcodes is identified and a representative cycle of the first significant persistent homology is returned. An orthonormal basis of the multiway data is computed. A harmonic representative is obtained by computing a projection of the representative cycle to an orthogonal complement, and a treatment plan is determined based on the harmonic representation and the treatment plan is output to a user.

### BACKGROUND

The present invention generally relates to determining potential treatment plans for a medical condition using harmonic homology.

Multiway interactions in data occur in data sets where any given element is directly affected by multiple distinct and independent factors. One example of a multiway interaction is in the field of medical treatment plans. A given medical condition can be treated with varying treatment plans. The effectiveness of each possible treatment plan is impacted by a complex set of interactions between different factors. The complex interplay of features can make determining an ideal initial treatment plan difficult, resulting in potential time and expense being wasted on less effective initial treatment plans for a specific individual exhibiting a unique set of factors. Complex interplay between the different factors and the effectiveness of a potential treatment plan is referred to as multiway interactions in a data set.

Traditionally, mapping multiway interactions in a data set requires the assistance of statistical tools to help measure the effects of single factors, and to narrow in on individual factors. The existing tools are computationally expensive, and memory expensive for computing systems.

### SUMMARY

Embodiments of the present invention are directed to a computer-implemented method for using harmonic homology to disentangle multiway interactions. A non-limiting example of the computer-implemented method includes receiving an input set of multiway data. The multiway data includes a numerical representation of each factor in a set of interconnected factors affecting an outcome and each factor has a codependency on at least one other factor in the set of interconnected factors. A set of persistent homology barcodes is determined based on the multiway data using a processer. At least a first significant persistent homology barcode in the determined set of persistent homology barcodes is identified and a representative cycle of the first significant persistent homology is returned. An orthonormal basis of the multiway data is computed. A harmonic representative is obtained by computing a projection of the representative cycle to an orthogonal complement, and a predictive setup for an experiment is determined based on the harmonic representation and outputting the predictive setup for the experiment to a user.

Embodiments of the present invention are also directed to a computer system and a computer program product for implementing the computer implemented method.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts one exemplary process for identifying a potential treatment plan from a multiway interaction dataset using harmonic homology.

FIG. 2 visually depicts determining a persistent homology and identifying a most significant bar portions of the process of FIG. 1.

FIGS. 3, 4, 5, 6, and 7 depict a process of identifying a representative cycle and identifying a harmonic representative based on an orthonormal basis in the process of FIG. 1.

FIG. 8 depicts a generalized workflow of the process of FIG. 1.

FIG. 9 illustrates a computing environment including an example of an environment for the execution of at least some of the computer code involved in performing the inventive methods.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the disclosed embodiments, the various elements illustrated in the figures are provided with two or three digit reference numbers. With minor exceptions, the leftmost digit(s) of each reference number correspond to the figure in which its element is first illustrated.

### DETAILED DESCRIPTION

A computer-implemented method includes receiving at a processor an input set of multiway data. The multiway data includes a numerical representation of each factor in a set of interconnected factors affecting an outcome. Each factor has a codependency on at least one other factor in the set of interconnected factors. The interconnected factors include relevant genetic markers, environmental conditions, comorbidities, time since onset of condition, and severity. The method determines a set of persistent homology barcodes based on the multiway data using the processer. The method identifies at least a first significant persistent homology barcode in the set of persistent homology barcodes and returns a representative cycle of the first significant persistent homology and computing an orthonormal basis for a boundary of the multiway data. The method obtains a harmonic representative by computing the projection of the representative cycle to an orthogonal complement. The method determines a treatment plan based on the harmonic representation and outputs the treatment plan to a user. Use of the computer implemented method allows for a quicker and more accurate identification of a potential treatment plan from a large multiway data set, thereby reducing a number of potential treatment options within a treatment plan by reducing the number of redundant and/or not-beneficial treatments being implemented.

In an example, the method includes executing the treatment plan. Execution of the treatment plan provides for a quicker potential improvement of a patient, and allows for validation of the computer implemented method.

In another example, determining the set of persistent homology barcodes includes building a combinatorial structure using the input set of multiway data using the processor. By building a combinatorial structure from the multiway data the method is able to more quickly identify the connectedness of the data points within the multiway data.

In another example, the combinatorial structure is one of a Vietoris-Rips structure, an alpha structure, and a Czech complex. Each of the combinatorial structures provides statistical analysis benefits that can more accurately and quickly be analyzed using the computer-implemented method.

In another example, the combinatorial structure forms a simplical complex. The simplical complex provides a chart of the connectedness of the datapoints within the multiway data set, allowing the computer implemented method to more accurately identify and determine the persistent homology barcodes.

In another example, identifying at least a first significant persistent homology barcode in the set of persistent homology barcode includes identifying a longest persistent homology barcode in the set of persistent homology barcodes. Usage of at least one significant persistent homology barcode provides a basis to begin the analysis without requiring every homology barcode to be analyzed, thereby saving computer resources and increasing the speed of the computer-implemented method.

In another example, identifying at least a first significant persistent homology barcode in the set of persistent homology barcode includes identifying each bar above a quantile threshold of longest bars. Identification of the longest bar, a starting point of a bar, and/or a bar positioned in the lowest density region of bars allows the method to identify and isolate specific bars on which to base the analysis, thereby increasing the speed and accuracy of the experimental setup identification.

A computing system includes a processor and a non-transitory computer readable memory storing instructions for causing the processor to perform a method including receiving at a processor an input set of multiway data. The multiway data includes a numerical representation of each factor in a set of interconnected factors affecting an outcome. Each factor has a codependency on at least one other factor in the set of interconnected factors. The interconnected factors include relevant genetic markers, environmental conditions, comorbidities, time since onset of condition, and severity. The method determines a set of persistent homology barcodes based on the multiway data using the processer. The method identifies at least a first significant persistent homology barcode in the set of persistent homology barcodes and returns a representative cycle of the first significant persistent homology and computing an orthonormal basis for a boundary of the multiway data. The method obtains a harmonic representative by computing the projection of the representative cycle to an orthogonal complement. The method determines a treatment plan based on the harmonic representation and outputs the treatment plan to a user. Provision of a computer system incorporating the method provides an implementation tool which aides operators in determining an efficient initial setup for an experiment.

A computer program product includes a non-transitory computer readable storage medium storing instructions for causing a computer system to perform the method of A computer-implemented method includes receiving at a processor an input set of multiway data. The multiway data includes a numerical representation of each factor in a set of interconnected factors affecting an outcome. Each factor has a codependency on at least one other factor in the set of interconnected factors. The interconnected factors include relevant genetic markers, environmental conditions, comorbidities, time since onset of condition, and severity. The method determines a set of persistent homology barcodes based on the multiway data using the processer. The method identifies at least a first significant persistent homology barcode in the set of persistent homology barcodes and returns a representative cycle of the first significant persistent homology and computing an orthonormal basis for a boundary of the multiway data. The method obtains a harmonic representative by computing the projection of the representative cycle to an orthogonal complement. The method determines a treatment plan based on the harmonic representation and outputs the treatment plan to a user.

Existing computer systems apply statistical models to the multiway data to attempt to identify potential interactions and eliminate negative interactions. While these systems can extract sufficient data to create a general treatment plan, the identified treatment plans can frequently include unnecessary procedures or medications and/or redundant procedures or medications. Further, the statistical methods can take substantial amounts of time and computational resources to identify the potential treatment plans. Even with computerized and/or neural network based assistance the tools required the calculation times can be excessively long, and the resultant outputs are frequently not accurate enough to provide value.

In a more detailed example, most diseases and syndromes represent a highly complex set of interactions between different biological entities potentially operating across biological scales. These are also known as polygenic traits (alternatively referred to as “polygenes”). Polygenes give the advantage of producing a wider spectrum of phenotypic and genotypic variations in the population. An example of a polygenic condition that may lead to disease is type-2 diabetes which is impacted by multiple factors including varied genetic markers and varied environmental factors. Apart from type-2 diabetes, other examples of polygenic conditions that are medically important are hypertension, coronary heart disease, cancer, arthritis, and mental illness.

Traditionally, mapping polygenes to identify a treatment plan requires statistical tools, such as persistent homology barcodes, to help measure the effects of polygenes as well as narrow in on single genes. The initial statistical mapping is used to narrow treatment plans down by reducing or eliminating redundant treatment options identifying and removing less efficient or effective combinations of treatments, and thereby substantially reducing the risk of expensive, unnecessary, or redundant treatments, as well as increasing the effectiveness of a given treatment plan. Using only persistent homology barcodes can result in redundant and incomplete data sets and a less effective treatment plan, which in turn can result in a more expensive and time-consuming treatment plan.

In order to reduce the computational and memory expense and increase the effectiveness of the identified treatment plan, the present disclosure describes a computerized method that provides a way to identify interactions at different scales for polygenic phenotypes based on the topological structure of the data in high dimensional space using algebraic topology and persistent homology via harmonic representatives.

In some specific embodiments, the method includes receiving an input set of multiway data. The multiway data includes a numerical representation of each factor in a set of interconnected factors affecting an outcome and each factor has a codependency on at least one other factor in the set of interconnected factors. A set of persistent homology barcodes is determined based on the multiway data using a processer. At least a first significant persistent homology barcode in the determined set of persistent homology barcodes is identified and a representative cycle of the first significant persistent homology is returned. An orthonormal basis of the multiway data is computed. A harmonic representative is obtained by computing a projection of the representative cycle to an orthogonal complement, and a predictive setup for an experiment is determined based on the harmonic representation and outputting the predictive setup for the experiment to a user.

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” may be understood to include any integer number greater than or equal to one, i.e. one, two, three, four, etc. The terms “a plurality” may be understood to include any integer number greater than or equal to two, i.e. two, three, four, five, etc. The term “connection” may include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 900 contains an example of an environment for the execution of at least some of the computer code involved in performing the inventive methods, such as “treatment plan identification code” 950. In addition to block 950, computing environment 900 includes, for example, computer 901, wide area network (WAN) 902, end user device (EUD) 903, remote server 904, public cloud 905, and private cloud 906. In this embodiment, computer 901 includes processor set 910 (including processing circuitry 920 and cache 921), communication fabric 911, volatile memory 912, persistent storage 913 (including operating system 922 and block 950, as identified above), peripheral device set 914 (including user interface (UI), device set 923, storage 924, and Internet of Things (IoT) sensor set 925), and network module 915. Remote server 904 includes remote database 930. Public cloud 905 includes gateway 940, cloud orchestration module 941, host physical machine set 942, virtual machine set 943, and container set 944.

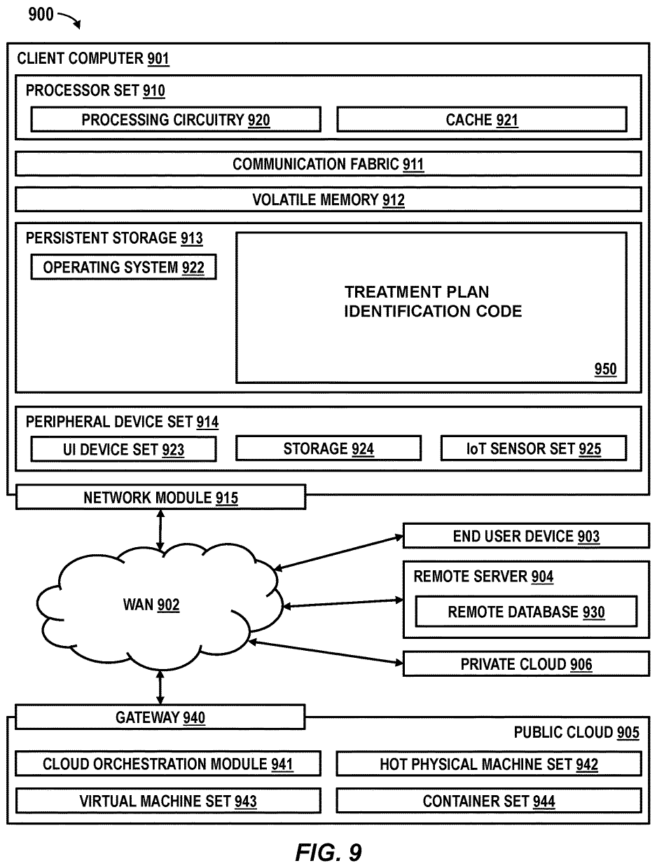

COMPUTER 901 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 930. As is well understood in the art of computer technology, and depending upon the technology, performance of a computer-implemented method may be distributed among multiple computers and/or between multiple locations. On the other hand, in this presentation of computing environment 900, detailed discussion is focused on a single computer, specifically computer 901, to keep the presentation as simple as possible. Computer 901 may be located in a cloud, even though it is not shown in a cloud in FIG. 9. On the other hand, computer 901 is not required to be in a cloud except to any extent as may be affirmatively indicated.

PROCESSOR SET 910 includes one, or more, computer processors of any type now known or to be developed in the future. Processing circuitry 920 may be distributed over multiple packages, for example, multiple, coordinated integrated circuit chips. Processing circuitry 920 may implement multiple processor threads and/or multiple processor cores. Cache 921 is memory that is located in the processor chip package(s) and is typically used for data or code that should be available for rapid access by the threads or cores running on processor set 910. Cache memories are typically organized into multiple levels depending upon relative proximity to the processing circuitry. Alternatively, some, or all, of the cache for the processor set may be located “off chip.” In some computing environments, processor set 910 may be designed for working with qubits and performing quantum computing.

Computer readable program instructions are typically loaded onto computer 901 to cause a series of operational steps to be performed by processor set 910 of computer 901 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 921 and the other storage media discussed below. The program instructions, and associated data, are accessed by processor set 910 to control and direct performance of the inventive methods. In computing environment 900, at least some of the instructions for performing the inventive methods may be stored in block 950 in persistent storage 913.

COMMUNICATION FABRIC 911 is the signal conduction paths that allow the various components of computer 901 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 912 is any type of volatile memory now known or to be developed in the future. Examples include dynamic type random access memory (RAM) or static type RAM. Typically, the volatile memory is characterized by random access, but this is not required unless affirmatively indicated. In computer 901, the volatile memory 912 is located in a single package and is internal to computer 901, but, alternatively or additionally, the volatile memory may be distributed over multiple packages and/or located externally with respect to computer 901.

PERSISTENT STORAGE 913 is any form of non-volatile storage for computers that is now known or to be developed in the future. The non-volatility of this storage means that the stored data is maintained regardless of whether power is being supplied to computer 901 and/or directly to persistent storage 913. Persistent storage 913 may be a read only memory (ROM), but typically at least a portion of the persistent storage allows writing of data, deletion of data and re-writing of data. Some familiar forms of persistent storage include magnetic disks and solid state storage devices. Operating system 922 may take several forms, such as various known proprietary operating systems or open source Portable Operating System Interface type operating systems that employ a kernel. The code included in block 950 typically includes at least some of the computer code involved in performing the inventive methods.

PERIPHERAL DEVICE SET 914 includes the set of peripheral devices of computer 901. Data communication connections between the peripheral devices and the other components of computer 901 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 923 may include components such as a display screen, speaker, microphone, wearable devices (such as goggles and smart watches), keyboard, mouse, printer, touchpad, game controllers, and haptic devices. Storage 924 is external storage, such as an external hard drive, or insertable storage, such as an SD card. Storage 924 may be persistent and/or volatile. In some embodiments, storage 924 may take the form of a quantum computing storage device for storing data in the form of qubits. In embodiments where computer 901 is required to have a large amount of storage (for example, where computer 901 locally stores and manages a large database) then this storage may be provided by peripheral storage devices designed for storing very large amounts of data, such as a storage area network (SAN) that is shared by multiple, geographically distributed computers. IoT sensor set 925 is made up of sensors that can be used in Internet of Things applications. For example, one sensor may be a thermometer and another sensor may be a motion detector.

NETWORK MODULE 915 is the collection of computer software, hardware, and firmware that allows computer 901 to communicate with other computers through WAN 902. Network module 915 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 915 are performed on the same physical hardware device. In other embodiments (for example, embodiments that utilize software-defined networking (SDN)), the control functions and the forwarding functions of network module 915 are performed on physically separate devices, such that the control functions manage several different network hardware devices. Computer readable program instructions for performing the inventive methods can typically be downloaded to computer 901 from an external computer or external storage device through a network adapter card or network interface included in network module 915.

WAN 902 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 903 is any computer system that is used and controlled by an end user (for example, a customer of an enterprise that operates computer 901), and may take any of the forms discussed above in connection with computer 901. EUD 903 typically receives helpful and useful data from the operations of computer 901. For example, in a hypothetical case where computer 901 is designed to provide a recommendation to an end user, this recommendation would typically be communicated from network module 915 of computer 901 through WAN 902 to EUD 903. In this way, EUD 903 can display, or otherwise present, the recommendation to an end user. In some embodiments, EUD 903 may be a client device, such as thin client, heavy client, mainframe computer, desktop computer and so on.

REMOTE SERVER 904 is any computer system that serves at least some data and/or functionality to computer 901. Remote server 904 may be controlled and used by the same entity that operates computer 901. Remote server 904 represents the machine(s) that collect and store helpful and useful data for use by other computers, such as computer 901. For example, in a hypothetical case where computer 901 is designed and programmed to provide a recommendation based on historical data, then this historical data may be provided to computer 901 from remote database 930 of remote server 904.

PUBLIC CLOUD 905 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 905 is performed by the computer hardware and/or software of cloud orchestration module 941. The computing resources provided by public cloud 905 are typically implemented by virtual computing environments that run on various computers making up the computers of host physical machine set 942, which is the universe of physical computers in and/or available to public cloud 905. The virtual computing environments (VCEs) typically take the form of virtual machines from virtual machine set 943 and/or containers from container set 944. It is understood that these VCEs may be stored as images and may be transferred among and between the various physical machine hosts, either as images or after instantiation of the VCE. Cloud orchestration module 941 manages the transfer and storage of images, deploys new instantiations of VCEs and manages active instantiations of VCE deployments. Gateway 940 is the collection of computer software, hardware, and firmware that allows public cloud 905 to communicate through WAN 902.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 906 is similar to public cloud 905, except that the computing resources are only available for use by a single enterprise. While private cloud 906 is depicted as being in communication with WAN 902, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 905 and private cloud 906 are both part of a larger hybrid cloud.

When attempting to identify potential treatment plans for medical conditions multiway data sets present a problem due to the complex interplay between the factors that impact, or otherwise alter, the effectiveness of a given treatment plan for the medical condition.

Turning now to an overview of the aspects of the invention, one or more embodiments of the invention address the above-described shortcomings of the prior art by utilizing persistent harmonic representatives to narrow down the barcodes. The persistent harmonic representatives allow for more nuanced prediction setups based on the original input features. The use of the harmonic representatives allows for a better interpretation of the features space, with the interpretation not being limited to a bar plot of the feature space. This in turn reduces the computational resources and the time required to identify the prediction setups.

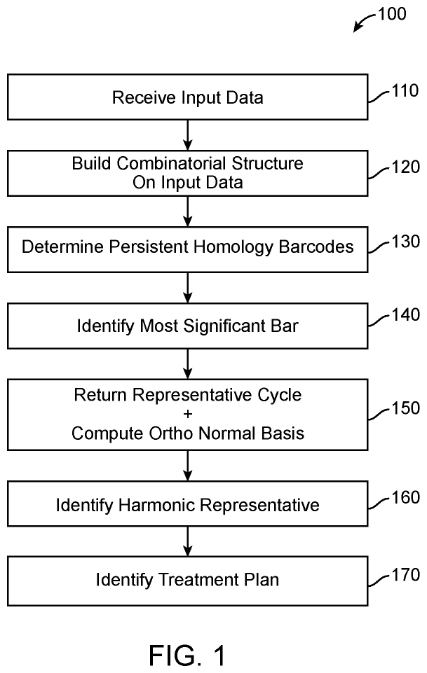

Turning now to a more detailed description of aspects of the present invention, FIG. 1 depicts a generalized process 100 for identifying and determining predictive setups using a multiway data set. As used herein predictive setups refer to a set of conditions or factors that, when combined in a certain manner, are expected to provide an indicator of a likely event. By way of example, when four particular genes are present within an individual the combination may provide a predictor of a certain percentage change of a medical condition such as Type I diabetes occurring. The predictive setup can then be used to establish an experiment or trial to determine the actual impact of each of the factors.

The generalized process 100 includes initially receiving a set of input multiway data at a “Receive Input Data” step 110. The multiway data set includes a number of events/conditions and factors associated with the event/condition. When planning a treatment plan, the multiway data can include any number of factors such as: relevant genetic markers, environmental conditions, comorbidities, time since onset of condition, severity, and any combination thereof.

A combinatorial structure is constructed on top of and using the received input data in a “Build Combinatorial Structure On Input Data” step 120. The combinatorial structure can be built using a Vietoris-Rips complex, an alpha complex, a Cech complex model, or any similar combinatorial structure.

Based on the combinatorial structure, a number of persistent homology barcodes are computed in a “Determine Persistent Homology Barcodes” step 130, and the most significant bars, of the computed persistent homology barcodes are identified in an “Identify Most Significant Bars” step 140. In some examples, the most significant bar is identified using rules determined via prior information and/or external system knowledge. Within the context of determining an optimal treatment plan, prior information and/or external knowledge can be derived from the expertise of a practitioner employing the process 10 as well as their personal experience treating the condition to which the process 100 is being applied. In some alternate examples, the set of all bars can be considered, rather than a subset of the most significant bars.

The process 100 then proceeds identify and return a representative cycle of each identified significant bar and compute an orthonormal basis for the combinatorial structure boundary at time (b) of the representative cycle in a “Return Representative Cycle+Computer Orthonormal Basis” step 150.

A harmonic representative of the representative cycle is obtained by computing the projection of the representative cycle onto the orthogonal complement of the boundary in an “Identify Harmonic Representative” step 160. Based on the projection, relevant causal factors in the multiway data set are extracted by analyzing the coefficients of the harmonic representative and the casual factors are correlated with identified treatments for a treatment plan in an “Identify Treatment Plan” step 170. In one particular example, weighted representative nodes/coefficients are directly used as an output during the step 170 and the weight of the nodes correlates to the value provided by a given part of the treatment plan. With each node corresponding to one treatment and the weight of each node corresponding to the impact of the treatment on a positive outcome. In another alternative, the weighted representative nodes/coefficients are processed as an input to a machine learning model and machine learning model identifies and outputs an optimal treatment plan based on the weighted representative nodes/coefficients.

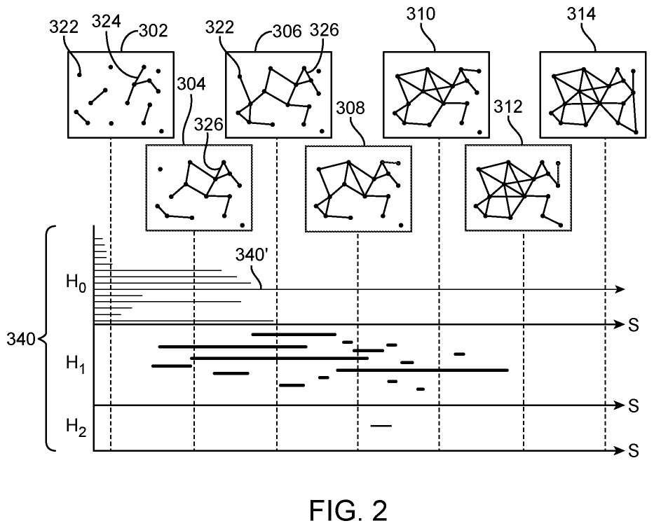

With continued reference to the process 100 of FIG. 1, FIG. 2 illustrates a simplified example of determining the homology barcodes and identifying a significant bar among the identified barcodes of steps 130 and 140 of the process 100. The combinatorial structures are constructed by plotting discrete data points in a set of plots 302, 304, 306, 308, 310, 312, 314 (referred to collectively as plots 300). The plots 300 include individual data points 322 with each plot 300 corresponding to a certain radii of connectedness. The lowest numeral plot 302 has the lowest radii of connectedness, and the highest numeral plot 314 has the highest radii of connectedness. An edge 324 is created connecting each data point 322 to each other data point 322 within its radii of connectedness. A bounded space 326 is created whenever three or more data points 322 (alternately referred to as nodes) intersect with each other such that the corresponding edges 324 form a closed space 326.

Conceptually, as the radii is increased across the plots 300, the complexes (interconnected bounded spaces 326 and edges 324) formed change, as do the homology groups created by the intersections. By varying the radii of the data pints 322, the topology or topologies that persist across multiple radii become apparent. The barcodes, 340 illustrated on the bottom chart represent the life span of a given connection (e.g., how long the connection persists) as the radii is increased with H0 representing connected components, H1 representing one dimensional cycles (similar to graph cycles), and H2 representing voids.

Once the barcodes 340 are generated, the most significant barcode, or most significant set of barcodes is selected according to the predetermined methodology. In one example, the longest barcode 340′ is selected. In another example, a quantile threshold can be imposed to select longest bars.

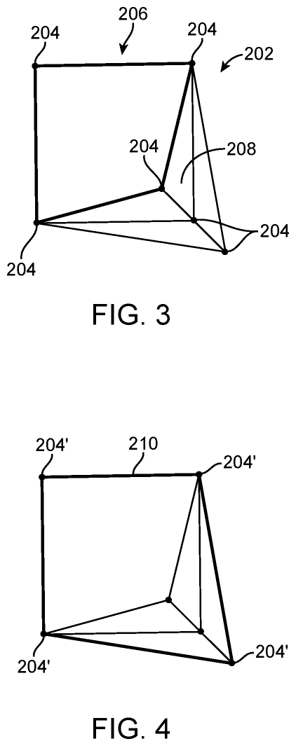

With continued reference to the process described at FIG. 1, FIGS. 3-7 visually represent a simplified example of steps 150 and 160 of the process 100 described at FIG. 1. It is appreciated that a practical example can include substantially larger numbers of nodes and substantially larger number of edges connecting the nodes, with an increased number of nodes exponentially increasing the computational complexity. At FIG. 3 a simplical complex 202 is built on top of the input data. The simplical complex 202 is a combinatorial object where the vertices (nodes 204) correspond to data points in the input data, and edges 206, triangles 208, and higher dimensional faces (e.g. shapes having more than three sides, omitted from the illustrated example for clarity of explanation) are added according to an application dependent distance function or proximity rule.

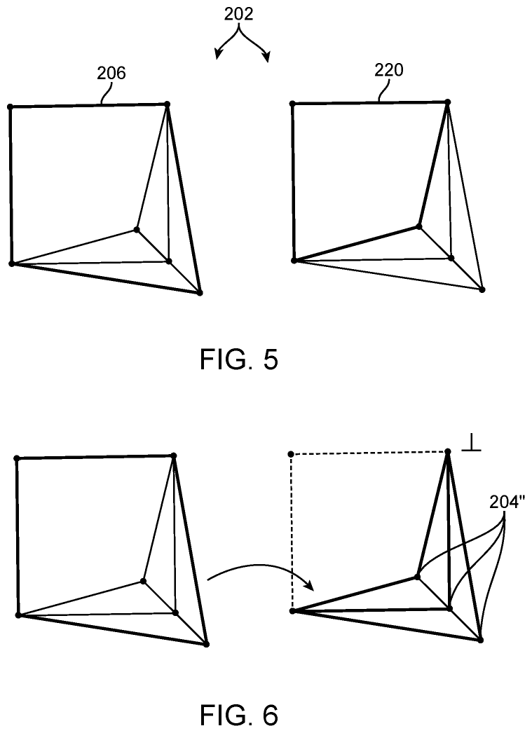

Using existing persistent homology algorithms, topological features, and representatives of the topological features defined by the simplicial complex 202 are extracted. One example feature 210 is highlighted in FIG. 4, which illustrates the simplical complex 202 of FIG. 3. The example feature 210 is a one-dimensional feature 210 defined by the four outermost nodes 204′, and the edges 206′ connecting the nodes 204′. One problem this feature 210 presents is illustrated in FIG. 5, which shows that the homology (e.g. general shape) of the feature 210 is the same as at least one other feature 220 in the simplical complex 202.

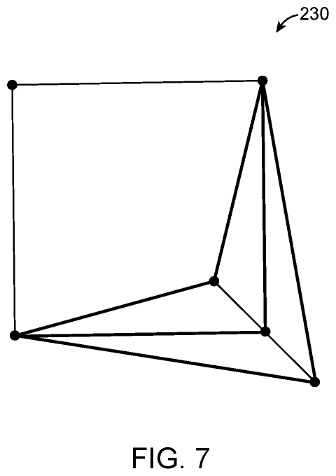

The process 100 address this problem by projecting the representative cycle 200 to the orthogonal complement of the boundary of the simplical complex 202. This is illustrated in FIG. 6, with the orthogonal complement being the shape defined by each edge 206″ connecting the vertices 204″. The projection results in a feature 230, illustrated at FIG. 7 that is a harmonic representative feature, and the projection of every distinct cycle that has the same homological class (e.g. is the same shape) will return the same harmonic cycle.

The representative harmonic cycle returns a weighted combination of all possible representatives of a given homology class, thereby eliminating the bias introduced by the choice of a specific harmonic cycle. Moreover, the important aspects of the cycle can be extracted by considering higher weighted simplices. This approach examines patterns in feature space and is not limited to extracting topological features which aids downstream data analysis. Further, this process 100 naturally integrates multiple types of data analysis including multi-omics, omics, clinical, and any combination thereof.

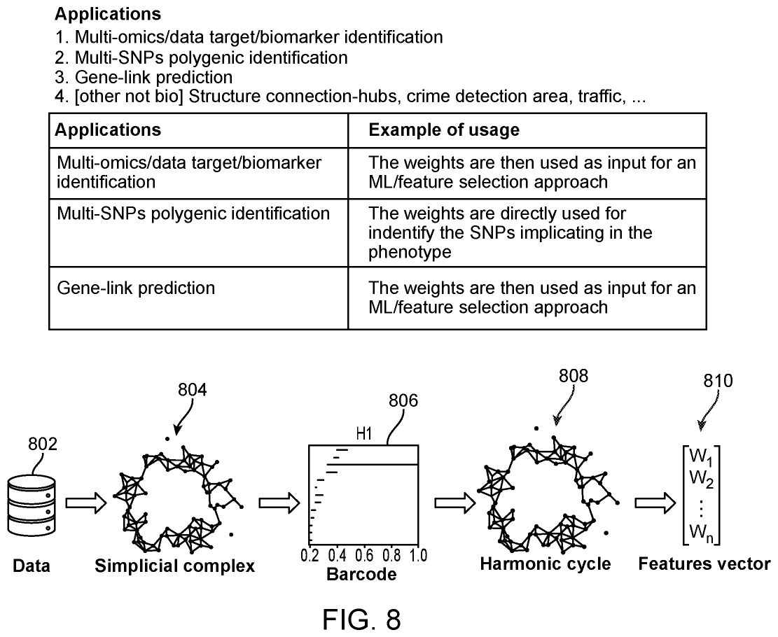

With continued reference to FIGS. 1-7, FIG. 8 visually represents one example of the full process 100, including receiving the input data at 802, generation of a simplical complex at 804, generation of the barcodes from the simplical complex at 806, identification of the representative harmonic cycle at 808 from the barcodes, and finally generation of the feature vectors representing extracted information at 810.

In the described application, the process 100 is applied to multi-omics data regarding a set of potential treatment options for a medical condition. The output of the process identifies the combination of treatment options that is expected to give an optimal result, while factoring in any comorbidities, and environmental factors that may influence the result.

In one practical implementation the process 100 is applied to (multi-) omics data regarding a given cell lines to identify bio-markers for a drug that may be closer to identified elements. The identified bio-markers are then retained for a drug development test phase, or provided to a patient, allow for a substantially reduction in the number of potentially viable drugs to be tested.

In another practical implementation, the process 100 is applied to a genome-wide assembly study (GWAS) with the process 10 being provided a series of plant GAWAS elements with a desired trait, and the process 100 identifies the SNP's interplay/importance and a cross plant is generated to maintain these SNPs. An SNP array is a type of DNA microarray which is used to detect polymorphisms within a population.

In yet another practical implementation, the process 100 is provided a series of microbiome data and identifies a microbe's interplay and abundance. The output of the process 100 suggests a best and relative amount of fertilizer, water, light and/or other factors to plan growth in an ideal condition.

Furthermore, the system can be applied to any similar data series while still falling within the disclosed process 100.

The present invention may be a system, a method, and/or a computer program product at any possible technical detail level of integration. The computer program product may include a computer readable storage medium (or media) having computer readable program instructions thereon for causing a processor to carry out aspects of the present invention.

As the modeling cannot practically be done within a human mind, or using a paper and pencil, due to the inherent complexity of multiway data sets, the process 100 is implemented using a computer based statistical modeling system. In such an example, the process is stored on a computer readable storage medium, such as a disk and/or within a computer memory,

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

What is claimed is:

1. A computer-implemented method comprising:
receiving at a processor an input set of multiway data, wherein the multiway data includes a numerical representation of each factor in a set of interconnected factors affecting an outcome, and wherein each factor has a codependency on at least one other factor in the set of interconnected factors, and wherein the interconnected factors include relevant genetic markers, environmental conditions, comorbidities, and time since onset of condition, severity;
determining a set of persistent homology barcodes based on the multiway data using the processer;
identifying at least a first significant persistent homology barcode in the set of persistent homology barcodes, returning a representative cycle of the first significant persistent homology and computing an orthonormal basis for a boundary of the multiway data;
obtaining a harmonic representative by computing the projection of the representative cycle to an orthogonal complement; and
determining a treatment plan based on the harmonic representation and outputting the treatment plan to a user.

2. The computer implemented method of claim 1, further comprising executing the treatment plan.

3. The computer implemented method of claim 1, wherein determining the set of persistent homology barcodes includes building a combinatorial structure using the input set of multiway data using the processor.

4. The computer implemented method of claim 3, wherein the combinatorial structure is one of a Vietoris-Rips structure, an alpha structure, and a Czech complex.

5. The computer implemented method of claim 4, wherein the combinatorial structure forms a simplical complex.

6. The computer implemented method of claim 1, wherein identifying at least a first significant persistent homology barcode in the set of persistent homology barcode includes identifying a longest persistent homology barcode in the set of persistent homology barcodes.

7. The computer implemented method of claim 1, wherein identifying at least a first significant persistent homology barcode in the set of persistent homology barcode includes identifying each bar above a quantile threshold of longest bars.

8. A computing system comprising:
a processor and a non-transitory computer readable memory storing instructions for causing the processor to perform the method of:
receiving at the processor an input set of multiway data, wherein the multiway data includes a numerical representation of each factor in a set of interconnected factors affecting an outcome, and wherein each factor has a codependency on at least one other factor in the set of interconnected factors, and wherein the interconnected factors include relevant genetic markers, environmental conditions, comorbidities, and/or time since onset of condition, severity;
determining a set of persistent homology barcodes based on the multiway data using the processer;
identifying at least a first significant persistent homology barcode in the set of persistent homology barcodes, returning a representative cycle of the first significant persistent homology and computing an orthonormal basis for a boundary of the multiway data;
obtaining a harmonic representative by computing the projection of the representative cycle to an orthogonal complement; and
determining a treatment plan based on the harmonic representation and outputting the treatment plan to a user.

9. The computer implemented method of claim 8, further comprising executing the treatment plan.

10. The computer implemented method of claim 8, wherein determining the set of persistent homology barcodes includes building a combinatorial structure using the input set of multiway data using the processor.

11. The computer implemented method of claim 10, wherein the combinatorial structure is one of a Vietoris-Rips structure, an alpha structure, and a Czech complex.

12. The computer implemented method of claim 11, wherein the combinatorial structure forms a simplical complex.

13. The computer implemented method of claim 8 wherein identifying at least a first significant persistent homology barcode in the set of persistent homology barcode includes identifying a longest persistent homology barcode in the set of persistent homology barcodes.

14. The computer implemented method of claim 8, wherein identifying at least a first significant persistent homology barcode in the set of persistent homology barcode includes identifying a longest barcode and/or each bar above a quantile threshold of longest bars.

15. A computer program product comprising:
a non-transitory computer readable storage medium storing instructions for causing a computer system to perform the method of:
receiving at a processor an input set of multiway data, wherein the multiway data includes a numerical representation of each factor in a set of interconnected factors affecting an outcome, and wherein each factor has a codependency on at least one other factor in the set of interconnected factors, and wherein the interconnected factors include relevant genetic markers, environmental conditions, comorbidities, and/or time since onset of condition, severity;
determining a set of persistent homology barcodes based on the multiway data using the processer;
identifying at least a first significant persistent homology barcode in the set of persistent homology barcodes, returning a representative cycle of the first significant persistent homology and computing an orthonormal basis for a boundary of the multiway data;
obtaining a harmonic representative by computing the projection of the representative cycle to an orthogonal complement; and
determining a treatment plan based on the harmonic representation and outputting the treatment plan to a user.

16. The computer program product of claim 15, wherein the operations further comprise executing the treatment plan.

17. The computer program product of claim 15, wherein determining the set of persistent homology barcodes includes building a combinatorial structure using the input set of multiway data using the processor.

18. The computer program product of claim 17, wherein the combinatorial structure is one of a Vietoris-Rips structure, an alpha structure, and a Czech complex.

19. The computer program product of claim 18, wherein the combinatorial structure forms a simplical complex.

20. The computer program product of claim 18, wherein identifying at least a first significant persistent homology barcode in the set of persistent homology barcode includes identifying a longest persistent homology barcode in the set of persistent homology barcodes.
